(19) United States
(12) Patent Application Publication
(10) Pub. No.: US 2026/0216519 A1
(43) Pub. Date: Jul. 30, 2026

(54) INTEGRATING BIOIMPEDANCE MEASUREMENT INTO AN IMPLANTABLE PULSE GENERATOR FOR HEART RATE AND RESPIRATORY DETECTION

(71) Applicant: The Alfred E. Mann Foundation for Scientific Research, Valencia, CA (US)

(72) Inventors: Anthony C. Ng, Santa Clarita, CA (US); Rankiri Tissa Karunasiri, Valencia, CA (US)

(21) Appl. No.: 19/457,526

(22) Filed: Jan. 23, 2026

Related U.S. Application Data
(60) Provisional application No. 63/751,708, filed on Jan. 30, 2025.

(51) Int. Cl. A61N 1/36; A61N 1/05

(52) U.S. Cl. CPC A61N 1/36139; A61N 1/0553; A61N 1/0556; A61N 1/36125; A61N 1/3614; A61N 1/36157; A61N 1/36171

(57) ABSTRACT

An implantable medical system includes a lead, distal electrodes, and a medical device. The medical device includes a housing, a surface electrode, an AC current source, a multipole analog switch, a voltmeter, and a controller. The controller is configured to operate the multipole analog switch to selectively change the medical system between a neurostimulation mode and a bioimpedance measurement mode. In the neurostimulation mode, AC current is delivered from the AC current source and through the lead to a first distal electrode and returned from a second distal electrode to the medical device through the lead. In the bioimpedance measurement mode, AC current is delivered from the AC current source through the lead to a third distal electrode and the voltmeter measures a voltage between the third distal electrode and the surface electrode on the housing.

### CROSS-REFERENCE TO RELATED APPLICATION(S)

The present application claims priority to and the benefit of U.S. Provisional Application No. 63/751,708, filed Jan. 30, 2025 in the United States Patent and Trademark Office, the entire content of which is incorporated herein by reference.

### BACKGROUND

### 1. Field

The present disclosure relates to implantable pulse generators.

### 2. Description of the Related Art

Implantable pulse generation (IPG) devices utilize electrical nerve stimulation to treat a variety of medical conditions in a patient, such as epilepsy, depression, and obstructive sleep apnea. The electrical nerve stimulation may be timed or synchronized with the patient's breathing (i.e., the patient's respiratory cycle). Some related art systems and methods measure the patient's breathing utilizing an implanted pressure sensor or an inertial measurement unit (IMU). However, implanted pressure sensors and IMUs frequently fail to detect every respiratory cycle due to other artifacts in the signal and/or a low signal-to-noise ratio.

The above information disclosed in this Background section is only for enhancement of understanding of the background of the invention and therefore it may contain information that does not constitute prior art.

### SUMMARY

The present disclosure relates to various embodiments of an implantable medical system. In one embodiment, the implantable medical system includes a lead having a proximal end and a distal end, distal electrodes coupled to the distal end of the lead, and a medical device coupled to the proximal end of the lead. The medical device includes a housing, at least one surface electrode on the housing, an AC current source in the housing, a multipole analog switch in the housing coupled to the AC current source, a voltmeter in the housing having a first input coupled to a first distal electrode via the lead, and a second input coupled to the at least one surface electrode on the housing; and a controller in the housing coupled to the multipole analog switch. The controller is configured to operate the multipole analog switch to selectively change the medical system between a neurostimulation mode and a bioimpedance measurement mode. In the neurostimulation mode, AC current is delivered from the AC current source and through the lead to a first distal electrode and returned from a second distal electrode to the medical device through the lead. In the bioimpedance measurement mode, AC current is delivered from the AC current source through the lead to a third distal electrode and the voltmeter measures a voltage between the third distal electrode and the at least one surface electrode on the housing.

In the bioimpedance measurement mode, the controller may be configured to determine an impedance based on the voltage sensed by the voltmeter.

In the bioimpedance measurement mode, the third distal electrode may not be configured for neurostimulation.

In the bioimpedance measurement mode, the controller may be configured to determine a heart rate based on variations in the impedance.

In the bioimpedance measurement mode, the controller may be configured to determine parameters of a neurostimulation protocol based on the heart rate.

In the neurostimulation mode, the first distal electrode and the second distal electrode may not be configured for sensing of the voltage.

In the bioimpedance measurement mode, the controller may be configured to determine a respiratory cycle based on variations in the impedance.

The respiratory cycle may include inspiratory and expiratory phases.

In the bioimpedance measurement mode, the controller may be configured to determine parameters of a neurostimulation protocol utilized in the neurostimulation mode based on the respiratory cycle.

The distal electrodes may include a nerve cuff, a helical cuff, paddle electrodes, or an electrode array.

The medical device may be configured for implantation in a thoracic region and the distal electrodes may be configured for implantation in a cervical region, or both the medical device and the distal electrodes may be configured for implantation in the thoracic region.

The first distal electrode, the second distal electrode, and the third distal electrode may not be arranged sequentially.

The AC current may have a frequency in a range from approximately 1 kHz to approximately 100 KHz.

The AC current may have an amplitude in a range from approximately 0.1 mA to approximately 5 mA.

In the bioimpedance measurement mode, the voltmeter may be configured to sample the voltage at a sampling rate of up to approximately 400 samples per minute.

In the bioimpedance measurement mode, the controller may be configured to de-noise the impedance utilizing an adaptive threshold algorithm.

The present disclosure also relates to various methods of operating an implantable medical system including a lead, distal electrodes, and a medical device implanted in a patient. In one embodiment, the method includes determining a bioimpedance of the patient by delivering AC current from an AC current source of the medical device through the lead to a third distal electrode; measuring, utilizing a voltmeter of the medical device, a voltage between the third distal electrode and at least one surface electrode on a housing of the medical device; and determining, by the implantable medical system, the bioimpedance of the patient based on the voltage. The method also includes neurostimulating the patient by delivering AC current from the AC current source through the lead to a first distal electrode and returning the AC current from a second distal electrode to the medical device through the lead. The neurostimulating is based on the bioimpedance of the patient.

The method may also include switching, utilizing time-multiplexing, between the determining of the bioimpedance of the patient and the neurostimulation of the patient.

The medical device may be implanted in a thoracic region of the patient and the distal electrodes may be implanted in a cervical region of the patient, or both the medical device and the distal electrodes may be implanted in a thoracic region of the patient.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter. One or more of the described features may be combined with one or more other described features to provide a workable implantable medical system or method of operating an implantable medical system.

### BRIEF DESCRIPTION OF THE DRAWINGS

The features and advantages of embodiments of the present disclosure will be better understood by reference to the following detailed description when considered in conjunction with the drawings. The drawings are not necessarily drawn to scale.

FIG. 1 is a schematic view of a nerve stimulation device according to one embodiment of the present disclosure implanted in a patient;

FIG. 2A is a schematic view of the nerve stimulation device of FIG. 1 operating in a nerve stimulation mode;

FIG. 2B is a schematic view of the nerve stimulation device of FIG. 1 operating in a bioimpedance measurement mode;

FIG. 3 is a schematic view of the nerve stimulation device of FIG. 1 performing a 4-point measurement of the voltage drop according to one embodiment of the present disclosure; and

FIG. 4 is a flowchart illustrating tasks of a method of treating a patient according to one embodiment of the present disclosure.

### DETAILED DESCRIPTION

Aspects of the present disclosure relate to various embodiments of a system and method for performing nerve stimulation (neurostimulation) to treat one or more medical issues of a patient, such as epilepsy, depression, and/or obstructive sleep apnea. The systems and methods are configured to determine in-vivo the patient's heart rate and respiratory cycle utilizing the existing metal can electrode and distal electrode(s) of a medical device, such as an implantable pulse generator (IPG), implanted in the patient, and to synchronize the electrical stimulation with the patient's respiratory cycle, which increases therapeutic efficacy and improves patient comfort. In one or more embodiments, the systems and methods of the present disclosure are configured to repurpose the existing distal electrode(s) of the medical device, which are utilized to deliver therapeutic stimulation to the patient, to measure bioimpedance and thereby determine or detect the patient's heart rate and respiration rate or cycle utilizing time-multiplexing with a switching network on the printed circuit board assembly (PCBA) of the medical device. Utilizing thoracic impedance as a respiratory signal and a cardiac signal to time the delivery of nerve stimulation may be more reliable and accurate than conventional methods of nerve stimulation utilizing an inertial measurement unit (IMU) and/or a pressure sensor because although an IMU may be able to detect heart rate based on small movements and acceleration caused by heart muscle contraction, IMU measurements are susceptible to noises generated from other unrelated body motions. By measuring bioimpedance that varies with electrical signals intrinsically generated by the heart near the heart during muscle contraction, the medical device eliminates such noise issues.

The terminology utilized herein is for the purpose of describing particular embodiments only and is not intended to be limiting of the present disclosure. As utilized herein, the singular forms “a”, “an” and “the” are intended to include the plural forms as well, unless the context clearly indicates otherwise. As utilized herein, the term “and/or” includes any and all combinations of one or more of the associated listed items.

It will be understood that, although the terms “first”, “second”, “third”, etc., may be utilized herein to describe one or more suitable elements, components, regions, and/or sections, these elements, components, regions, and/or sections should not be limited by these terms. These terms are only utilized to distinguish one element, component, region, layer or section from another element, component, region, layer or section. Thus, a first element, component, region, or section discussed could be termed a second element, component, region, or section, without departing from the spirit and scope of the present disclosure.

It will be understood that when an element is referred to as being “on”, “connected to”, “coupled to”, or “adjacent to” another element, it can be directly on, connected to, coupled to, or adjacent to the other element, or one or more intervening element(s) may be present. In contrast, when an element is referred to as being “directly on,” “directly connected to”, “directly coupled to”, or “immediately adjacent to” another element, there are no intervening elements present.

As utilized herein, the term “substantially” and similar terms are utilized as terms of approximation and not as terms of degree, and are intended to account for the inherent deviations in measured or calculated values that would be recognized by those of ordinary skill in the art. Also, the terms “about,” “approximately,” and similar terms, when utilized herein in connection with a numerical value or a numerical range, are inclusive of the stated value and refer to within an acceptable range of deviation for the particular value as determined by one of ordinary skill in the art, considering the measurement in question and the error associated with measurement of the particular quantity (e.g., the limitations of the measurement system).

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Example embodiments of the present disclosure will now be described with reference to the accompanying drawings. In the drawings, the same or similar reference numerals refer to the same or similar elements throughout. As utilized herein, the utilize of the term “may,” when describing embodiments of the present disclosure, refers to “one or more embodiments of the present disclosure.”

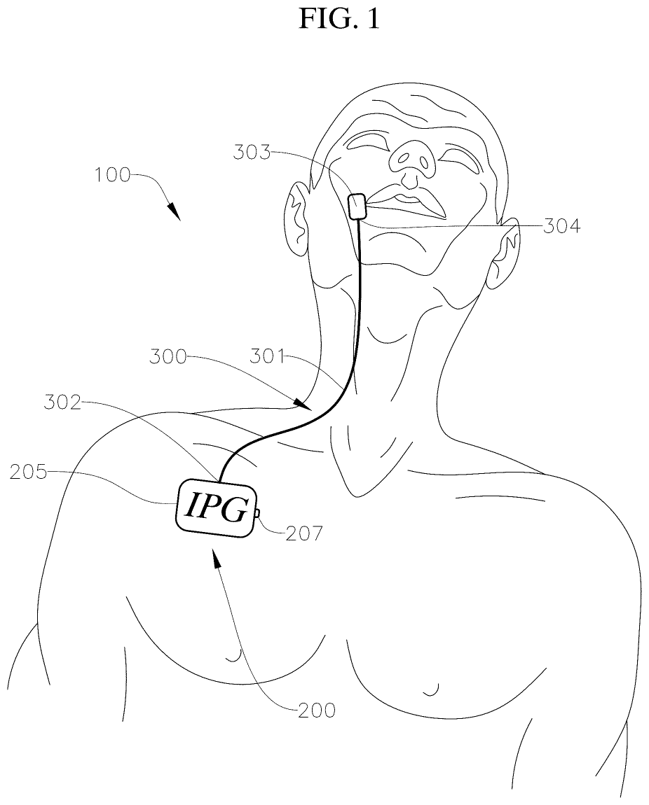

With reference now to FIG. 1, an implantable medical system (e.g., a nerve stimulation device) 100 for treating a patient according to one embodiment of the present disclosure includes a medical device (e.g., an implantable pulse generator (IPG)) 200 and a stimulation system 300 coupled to the medical device 200 for stimulating one or more nerves of the patient.

In the illustrated embodiment, the stimulation system 300 includes at least one implantable lead 301 having a proximal end 302 coupled to the medical device 200, and at least one distal electrode (e.g., at least one nerve cuff 303 comprising a plurality of distal electrodes 303(i)-303(iv)) coupled to a distal end 304 of the at least one implantable lead 301. The distal electrodes 303 may be provided on (or form) any suitable type or kind of electrode device, such as a nerve cuff, a helical cuff, paddle electrodes, or an electrode array. When the implantable medical system 100 is implanted in a patient, the medical device 200 may be implanted in the thorax (e.g., subcutaneously in the patient's chest, such as a subcutaneous pocket over the fascia of the pectoralis major muscle) and the distal electrodes 303 may be implanted in the cervical region of the patient below the head and above the shoulders (i.e., the medical device 200 may be implanted in the thoracic region of the patient and the distal electrodes 303 may be implanted in the cervical region of the patient). For instance, in one or more embodiments, the at least one implantable lead 301 may be tunneled between the chest wall and the neck over the patient's clavicle, and the distal electrodes 303 may contact (e.g., at least partially surround) the hypoglossal nerve (i.e., cranial nerve XII) or the Vagus nerve (i.e., cranial nerve X). In one or more embodiments, the medical device 200 may be implanted in any other suitable location, such as sub-pectorally in the patient. In one or more embodiments, both the medical device 200 and the distal electrodes 303 may be implanted in the thoracic region of the patient.

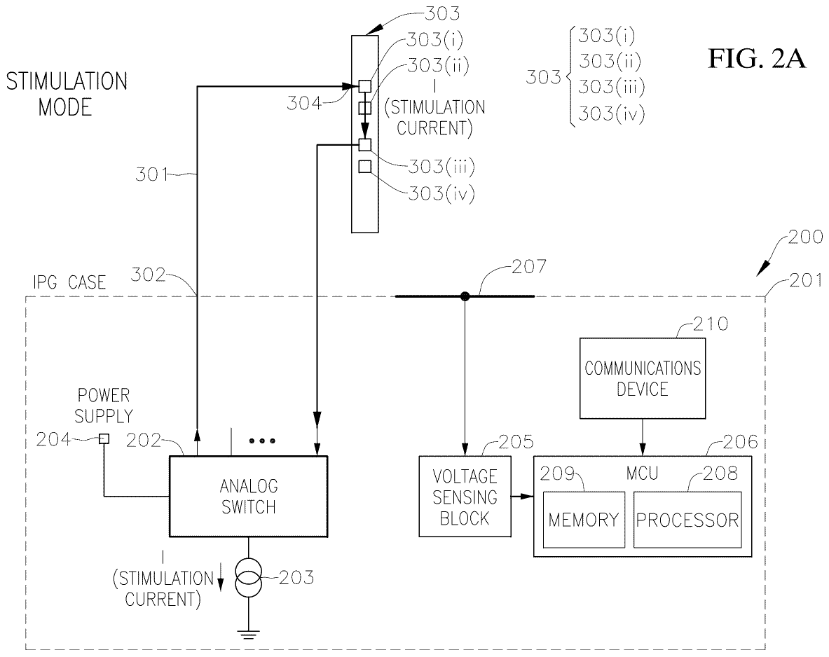

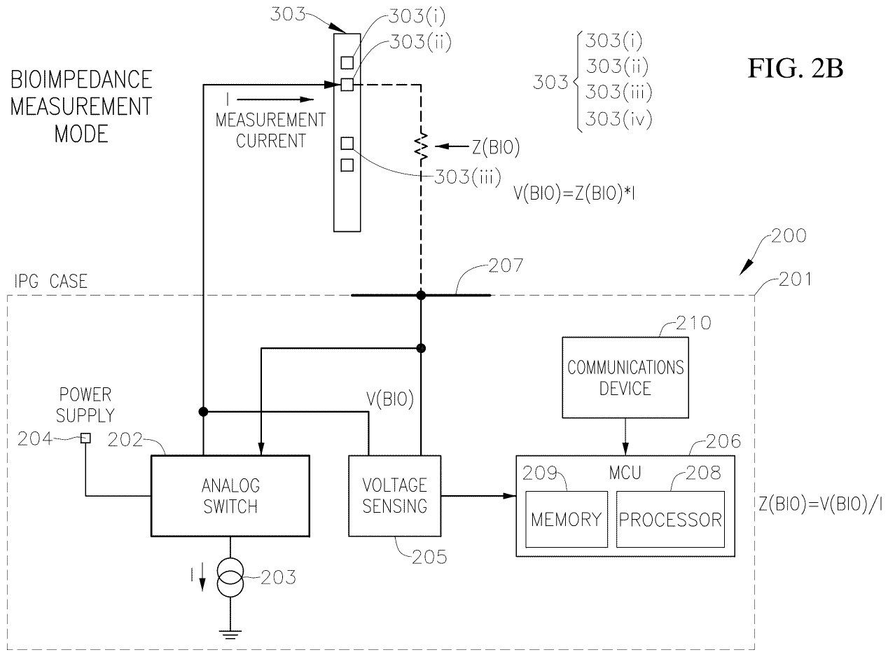

With reference now to the embodiment illustrated in FIG. 2A, the medical device 200 includes a case or housing 201, an analog switch (e.g., a multipole analog switch) 202 inside the case 201, a stimulation current source (e.g., an AC current source) 203 coupled to the analog switch 202, a power supply 204 coupled to the analog switch 202, a voltage sensing module or circuit (e.g., a voltmeter) 205, and a controller (e.g., a microcontroller unit (MCU)) 206 coupled to the voltage sensing circuit 205. The proximal end 302 of the implantable lead 301 extends through an opening in the case 201 and is connected to dedicated circuitry that provides stimulation pulses as controlled by the analog switch 202. Additionally, in the illustrated embodiment, the medical device 200 includes at least one surface electrode 207 (i.e., a can electrode) on the case 201.

In one or more embodiments, the controller 206 includes one or more processors (e.g., a processing circuit) 208 and a non-volatile memory device 209 (e.g., flash memory, ferroelectric random-access memory (FeRAM), magnetoresistive random-access memory (MRAM), phase-change memory (PCM), FeFET memory, and/or resistive random-access memory (RRAM)).

The term “processor” is utilized herein to include any combination of hardware, firmware, memory and software, employed to process data or digital signals. The hardware of a processor may include, for example, a microcontroller, application specific integrated circuits (ASICs), general purpose or special purpose central processors (CPUs), digital signal processors (DSPs), graphics processors (GPUs), and programmable logic devices such as field programmable gate arrays (FPGAs). In a processor, as utilized herein, each function is performed either by hardware configured, i.e., hard-wired, to perform that function, or by more general-purpose hardware, such as a CPU, configured to execute instructions stored in a non-transitory storage medium or memory. A processor may contain two or more processors, for example, a processor may include two processors, an FPGA and a CPU, interconnected on a PCB.

In one or more embodiments, the medical device 200 also includes a communications device 210 (e.g., a receiver and a transmitter, or a transceiver). The communications device 210 provides wireless communication links through the skin of the patient to a clinician programmer (CP) device and/or a patient remote (PR) device. Wireless links may include Bluetooth™, Bluetooth Low Energy or other protocols with suitable authentication and encryption to protect patient data.

The analog switch 202 is configured to change the implantable medical system between a stimulation mode (e.g., a neurostimulation mode) and a bioimpedance measurement mode. When the medical device 200 is operating in the stimulation mode, shown in FIG. 2A, the stimulation current/is delivered by the analog switch 202 to a selected one of the distal electrodes 303 (e.g., a first distal electrode 303(i) of the distal electrodes 303(i)-303(iv) in the nerve cuff) and the current is returned to the analog switch 202 through another one of the distal electrodes (e.g., a third distal electrode 303(iii) of the distal electrodes 303(i)-303(iv) in the nerve cuff). That is, in the stimulation mode, the analog switch 202 is configured to set which distal electrode is the anode and which distal electrode is the cathode and to deliver the stimulation current from the stimulation current source 203 to the anode and the cathode among the plurality of distal electrodes 303. In the stimulation mode, the system 300 may be utilized to provide nerve stimulation therapy to the patient for a variety of different medical conditions, such as epilepsy, depression, and/or obstructive sleep apnea (OSA). Additionally, when the medical device 200 is operating in the stimulation mode, the voltage sensing block is idle (inactive).

In the bioimpedance measurement mode, illustrated in FIG. 2B, the analog switch 202 is configured to deliver current I (having a known or fixed value in amps (A)) to one of the distal electrodes 303. However, unlike when the medical device 200 is operating in the stimulation mode (shown in FIG. 2A), the current returns to the medical device 200 through the tissue and the lung cavity of the patient, as depicted by the dashed lines in FIG. 2B. That is, the current delivered to the patient through the lead 301 and one of the distal electrodes 303 returns to the medical device 200 through the patient (e.g., the tissue and the lung cavity of the patient) rather than through the lead 301. In one or more embodiments, the distal electrode utilized in the bioimpedance measurement mode is different than the distal electrodes utilized in the stimulation mode. For instance, in one or more embodiments, first and third distal electrode 303(i) and 303(iii) may be utilized in the stimulation mode and the second distal electrode 303(ii) may be utilized in the bioimpedance measurement mode. In one or more embodiments, the medical device 200 is configured to switch from the stimulation mode (shown in FIG. 2A) to the bioimpedance measurement mode (shown in FIG. 2B) utilizing time-multiplexing (or time-domain multiplexing). Although in the illustrated embodiment the first, second, third, and fourth distal electrodes 303(i)-303(iv) are arranged sequentially (e.g., the first distal electrode 303(i) is the uppermost electrode, the second distal electrode 303(ii) is below the first distal electrode 303(i), the third distal electrode 303(iii) is below the second distal electrode 303(ii), and the fourth distal electrode 303(iv) is below the third distal electrode 303(iii) and is the lowermost electrode), in one or more embodiments the first, second, third, and fourth distal electrodes 303(i)-303(iv) may not be arranged sequentially. The terms “first,” “second,” “third,” and “fourth” in the first, second, third, and fourth distal electrodes 303(i)-303(iv) are used merely to distinguish one distal electrode from another distal electrode rather than to imply a position or location of the distal electrode relative to the other distal electrodes.

In one or more embodiments, in the bioimpedance measurement mode, the voltage sensing circuitry 205 is configured to measure or determine the voltage drop between the transmission of the current (I) from the analog switch 202 to one of the distal electrodes 303 and the return of the current through the patient to surface electrode 207 on the housing 201 of the medical device 200. In one or more embodiments, the voltage sensing circuitry 205 is configured to measure the voltage drop between (i) a line adjacent to the line that delivers the current to the distal electrode 303 and (ii) the return current at the surface electrode 207 on the case 201.

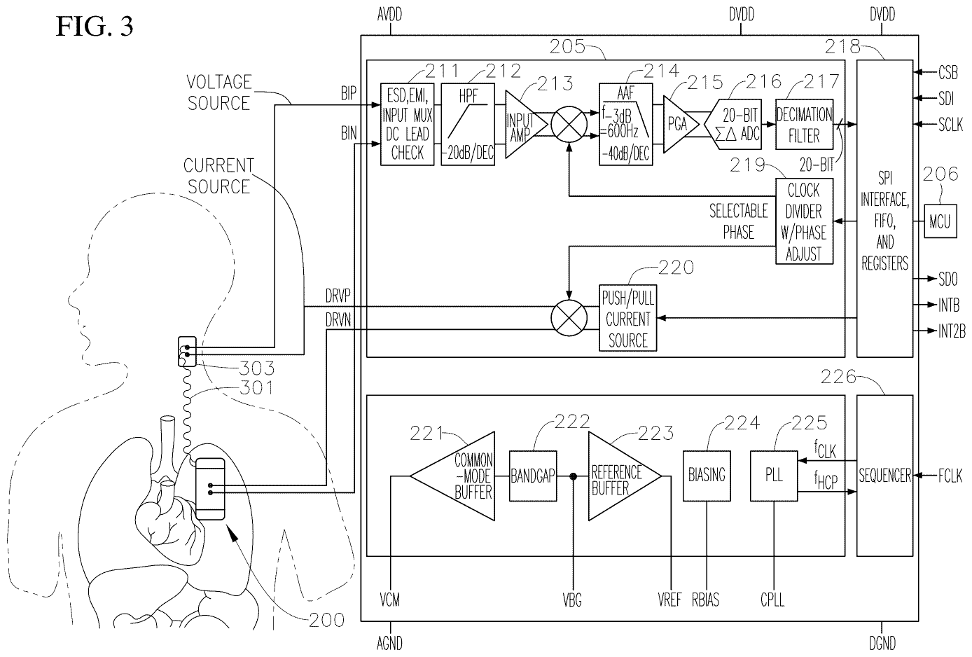

With reference now to FIG. 3, in one or more embodiments, the voltage sensing circuit 205 utilizes a 4-point measurement (i.e., a Kelvin measurement; a four-electrode measurement; or a tetrapolar measurement) to determine the voltage drop. Measuring the voltage drop in this manner is configured to eliminate voltage drop due to various components of the system (e.g., the resistance of the lead, the nerve cuff, the analog switch, and various metal traces), and thereby measure or determine the voltage drop due only to (or substantially only to) the bioimpedance of the patient. That is, the four-point measurement technique is configured to eliminate unwanted interface impedance at the electrodes and the impedance of the connecting lead that are not part of (or attributable to) the transthoracic impedance of the patient. As illustrated in FIG. 3, in one or more embodiments the voltage sensing circuitry 205 includes an input multiplexer 211 configured to receive input voltage sensing measurements from the medical device 200 and the distal electrodes 303, a high pass filter (HPF) 212 configured to receive the output from the input multiplexer 211, an input amplifier 213 configured to receive and amplify the output from the HPF 212, a low-pass filter 214 configured to receive the output from the amplifier 213, a programmable gain amplifier (PGA) 215 configured to receive and amplify the signal from the low-pass filter 214, an analog-to-digital (ADC) 216 configured to receive the output from the PGA 215, a decimation filter (i.e., a down-sampling filter) 217 configured to receive the output from the ADC 216, a serial peripheral interface (SPI) 218 configured to connect the output of the decimation filter 217 to the controller 206. Additionally, in the illustrated embodiment, the voltage sensing circuitry 205 includes a clock divider with phase adjust 219 connected to the controller 206 by the SPI 218 and configured to output a selectable phase to the low-pass filter 214. Furthermore, in the illustrated embodiment, the voltage sensing circuitry 205 includes a push/pull current source 220 connected to the SPI 218 and configured to deliver current to the medical device 200 and the distal electrodes 303.

With continued reference to FIG. 3, in one or more embodiments, the voltage sensing circuitry 205 may include support circuitry, such as, for example, a common-mode buffer 221, a bandgap circuit element 222 connected to the common-mode buffer 221, and a reference buffer 223 connected to the bandgap circuit element 222. Additionally, in one or more embodiments, the support circuitry may include a biasing circuit element 224 and a phase-locked circuit element 225 connected to a sequencer 226. In one or more embodiments, the voltage sensing circuitry 205 may be or include the Texas Instruments™ analog front end (AFE) 4500 device, the Analog Device™ MAX30002 device, or the Analog Device™ AD5933 device.

The voltage drop is a function of the bioimpedance of the patient and the current delivered by the distal electrodes 303 according to Equation 1 as follows:

$${V}_{Bio} = {Z}_{Bio} * I \qquad ( Equation 1 )$$

where the frequency-dependent bioimpedance, Z_{Bio}(f), includes a resistance vector R and a reactance vector X such that the bioimpedance Z_{Bio}(f)=R(f)+jX(f) and Z_{Bio}(f)²=R(f)²+X(f)². The resistance R(f) is a function of the ionic content of the patient's tissue and its geometry, and the reactance X(f) is a function of the polarization effects within the tissue.

The voltage sensing circuitry 205 may be the same as or similar to the analog device disclosed in “Ultra-Low-Power, Single-Channel Integrated Bioimpedance (BioZ) AFE,” the entire content of which is incorporated herein by reference.

The voltage drop (which is a function of the bioimpedance of the patient) that is measured or determined by the voltage sensing circuit 205 is transmitted to the controller 206. The controller 206 includes computer-executable (i.e., computer-readable) instructions which, when executed by a processor, cause the controller 206 to determine or calculate the bioimpedance (Z) from the voltage drop according to Equation 2 as follows:

$${Z}_{B i o} = \frac{{V}_{Bio}}{I} \qquad ( Equation 2 )$$

In one or more embodiments, in the thoracic impedance measured or determined by the controller 206 according to Equation 2 above, the frequency-dependent real (resistive) component, R(f), is in the range from approximately 15 Ohms (Ω) to approximately 45Ω and the imaginary component, X(f), has a phase angle of approximately 10°.

In one or more embodiments, the controller 206 may utilize an adaptive threshold algorithm to de-noise the bioimpedance signal and thereby increase the signal-to-noise ratio. A suitable adaptive threshold algorithm is described in Kamat (n.d.), Heart Rate Measurement using Bioimpedance Signal Analysis, Journal of Advanced Research in Electrical, Electronics and Instrumentation Engineering, available online at https://portuguese.rroij.com/peer-reviewed/a-heart-rate-measurement-using-bioimpedancesignal-analysis-42680.html, the entire content of which is incorporated herein by reference.

As described above, in one or more embodiments, different distal electrodes 303 may be utilized to perform neurostimulation and to determine the bioimpedance of the patient. However, in one or more embodiment, by utilizing time-multiplexing, the same distal electrode 303 may be utilized both to deliver therapeutic electrical stimulation (e.g., to the hypoglossal nerve or the Vagus nerve) and to deliver a current that is utilized to measure the bioimpedance of the patient, rather than relying upon separate dedicated electrodes for stimulation and voltage sensing.

The bioimpedance measured or determined by the controller 206 is a function of the patient's tissue and the volume of air in the patient's thoracic cavity, and thus the bioimpedance varies depending on whether the patient is inhaling or exhaling. As the patient's lung fill with air during inhalation, the electrical impedance of the thoracic cavity increases because air has a higher impedance than the surrounding tissues (e.g., blood and muscle). During exhalation, as air leaves the patient's lungs, the electrical impedance decreases. These changes in bioimpedance are due to the varying proportions of air and tissue within the path of the electrical current supplied by the analog switch 202 of the medical device 200 to the distal electrode 303. For instance, during inspiration, the patient's thoracic cage expands, which causes a decrease in bioimpedance. Conversely, during expiration, the patient's thoracic cage contracts, which causes an increase in bioimpedance.

The bioimpedance measured or determined by the controller 206 is also a function of the volume of blood in the patient's thoracic cavity, and thus the bioimpedance varies depending on whether the patient's heartbeat is in the contraction phase or the expansion phase. During the contraction phase of the heartbeat, there is a decrease in thoracic volume as the heart pumps blood out, leading to an increase in bioimpedance. Conversely, during the relaxation phase of the heartbeat, when the heart fills with blood again, there is a decrease in bioimpedance due to the increase in thoracic volume.

In the manner described above, the system 100 is configured to perform both bioimpedance pneumography (BIP) (i.e., electrical impedance pneumography (EIP)), which is the bioimpedance based on the patient's respiration, and bioimpedance cardiography (BIC) (i.e., electrical impedance cardiography (EIC)), which is the bioimpedance based on the patient's heartbeat. The system 100 may utilize these BIP to determine (or at least estimate) the patient's respiratory rate or inter-breath intervals (IBrI). Additionally, the system 100 may utilize the BIC to determine (or at least estimate) the patient's heart rate and/or cardiac output. In one or more embodiments, the controller 206 may be configured to calculate or otherwise determine the patient's heart rate by counting the number of pulses in the bioimpedance signal. Furthermore, in one or more embodiments, the controller 206 may be configured to remove, ignore, or reject signals common to the surface electrode 207 and the distal electrode 303 due to other body movements of the patient (i.e., common-mode noise) and thereby determine the patient's respiratory rate and heart rate independent of other body movements of the patient.

Additionally, in one or more embodiments, the system 100 is configured to perform both the BIP and BIC measurements in-vivo because the medical device 200 is implanted in the patient's thorax and the distal electrodes 303 are implanted on the hypoglossal nerve, the carotid sinus nerve, or the vagus nerve. Performing these measurements in-vivo has several benefits compared to related art non-invasive techniques that place electrodes on the surface of the patient's skin over the chest area, including: (i) enhanced signal quality because intrabody measurements provide more stable and consistent electrical impedance signals compared to external measurements, which can be affected by various factors, such as body position, breathing patterns, and muscle contractions; (ii) continuous monitoring of lung function and/or cardiac performance without the need for repeated external assessments, which provides valuable data for long-term patient care and management; (iii) improved accuracy regarding lung function and/or cardiac performance due to the absence of interference from external factors, the ability to measure directly at the site of interest, and the absence of additional skin resistance in the electrical path that is associated with taking external measurements; and (iv) increased patient comfort compared to repeated external assessments, particularly in cases where frequent testing is required.

In one or more embodiments, the AC current supplied by the current source 203 may be in the range from approximately 1 kHz to approximately 100 KHz. For instance, the AC current supplied by the current source 203 may be in a range from approximately 1 kHz to approximately 10 KHz to measure respiration rate and in a range from approximately 50 kHz to approximately 100 KHz to measure heart rate. Additionally, in one or more embodiments, the current supplied by the current source 203 may have a relatively low amplitude, such as in a range from approximately 0.1 mA to approximately 5 mA. Furthermore, in one or more embodiments, the voltage sensing circuitry 205 may have a sampling rate of up to approximately 400/minute. The respiratory rate in a normal human adult is 12-18 breaths per minute and the normal cardiac rate is 60-72 beats per minute (bpm), increasing to a maximum of about 200 bpm during exercise or disease, and a sampling rate up to approximately 400 samples per minute (i.e., approximately 7 samples per second) is sufficient to capture the relatively faster cardiac-related changes and the relatively slower respiratory changes of the patient.

The non-volatile memory device 202 of the medical device 200 includes computer-readable instructions which, when executed by the processor 201, cause the medical device 200 to deliver stimulation to the electrode 303 based on the thoracic impedance measured or determined by the voltage sensing circuit 205 and the MCU 206, as described above.

The non-volatile memory device 202 of the medical device 200 includes computer-readable instructions which, when executed by the processor 201, cause the medical device 200 to deliver stimulation to the distal electrodes 303 (e.g., the first distal electrode 303(i) and the second distal electrode 303(ii)) based on the thoracic impedance measured or determined by the voltage sensing circuit 205 and the controller 206. For example, in one or more embodiments, the computer-readable instructions, when executed by the processor 201, cause the medical device 200 to determine when the patient begins inhaling and stops inhaling (or begins exhaling) based on the values of the thoracic impedance. In general, the thoracic impedance changes (in both magnitude and direction) as the volume of the patient's lung changes and thus the thoracic impedance may be utilized to determine the patient's respiratory cycle (i.e., the thoracic impedance increases with increasing lung volume due to the presence of additional air in the lungs, and the thoracic impedance decreases with decreasing lung volume due the presence of less air in the lungs). Accordingly, in one or more embodiments, the computer-readable instructions, when executed by the processor 201, cause the medical device 200 to deliver stimulation to the distal electrode 303 (and, for example, the hypoglossal nerve, the Vagus nerve, the carotid sinus nerve, and/or the ansa cervicalis) in response to the value of the thoracic impedance indicating that the patient has started inhaling or is about to start inhaling (e.g., in response to the value of the thoracic impedance increasing from a minimum value), and cause the medical device 200 to cease delivering stimulation to the distal electrode 303 (and, for example, the hypoglossal nerve, the Vagus nerve, and/or the ansa cervicalis) in response to the value of the thoracic impedance indicating that the patient has stopped inhaling or started exhaling (e.g., in response to the value of the thoracic impedance reaching a maximum value or starting to decrease from a maximum value).

In one or more embodiments, the electrical stimulation delivered by the distal electrode 303 may treat any suitable medical condition afflicting the patient depending on the location of the distal electrode 303, such as epilepsy, depression, and/or obstructive sleep apnea (OSA). For instance, in one or more embodiments, the distal electrode 303 may at least partially surround the hypoglossal nerve of the patient and the electrical stimulation may be configured to treat obstructive sleep apnea experienced by the patient. The hypoglossal nerve innervates all the extrinsic and intrinsic muscles of the tongue except for the palatoglossus, which is innervated by the Vagus nerve. Accordingly, stimulating the hypoglossal nerve causes the patient's tongue to move forward, which relieves upper airway obstruction and thereby permits the patient to breath freely. In this manner, in one or more embodiments, the computer-readable instructions are configured to utilize the value of the thoracic impedance to control the timing of the delivery of the stimulation to the distal electrode 303 and the hypoglossal nerve (e.g., the computer-readable instructions, when executed by the processor 201, cause the medical device 200 to start and stop the delivery of the stimulation to the hypoglossal nerve based on the values of the thoracic impedance).

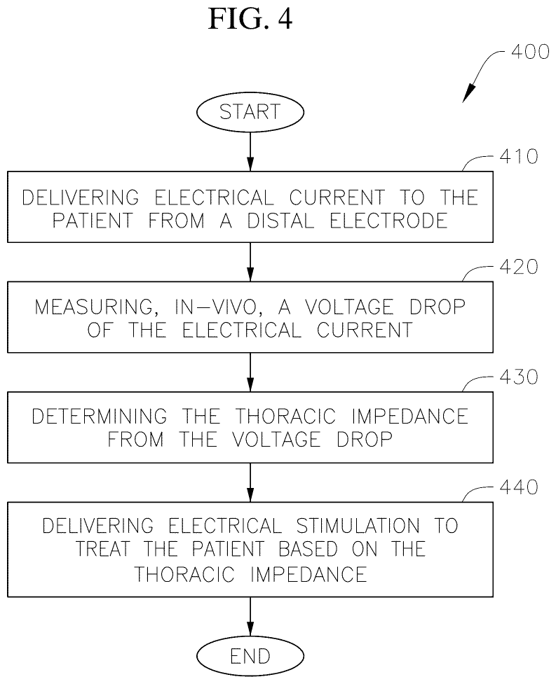

FIG. 4 is a flowchart illustrating tasks of a method 400 of operating an implantable medical system implanted in a patient. The method 400 may be performed, for example, to treat a patient afflicted by a medical condition, such as epilepsy, depression, hypertension, and/or obstructive sleep apnea (OSA). In the illustrated embodiment, the method 400 includes a task 410 of delivering electrical current to the patient from a distal electrode coupled via an implantable lead to a medical device (e.g., an implantable pulse generator (IPG)) implanted in the patient. In one or more embodiments, the distal electrode(s) may contact (e.g., at least partially surround) the hypoglossal nerve (i.e., cranial nerve XII), the Vagus nerve (i.e., cranial nerve X), or the ansa cervicalis of the patient. In one or more embodiments, the current supplied in task 410 may be alternating current (AC). In one or more embodiments, the AC current supplied by the current source may be in the range from approximately 1 kHz to approximately 100 KHz. Additionally, in one or more embodiments, the current supplied by the current source may have a relatively low amplitude, such as in a range from approximately 0.1 mA to approximately 5 mA.

In the illustrated embodiment, the method 400 also includes a task 420 of measuring, in-vivo, a voltage drop of the electrical current after the current returns to the can (i.e., housing or case) of the medical device through the patient (e.g., through the tissue and the lung cavity of the patient, rather than through the implantable lead that delivered the current to the distal electrode in task 410). The voltage drop may be measured in task 420 utilizing the distal electrode (which delivered the current in task 410) and a surface electrode on the can (housing) of the medical device implanted in the patient. In one or more embodiments, the task 420 may be performed by measuring or otherwise determining, utilizing voltage sensing circuitry in the medical device, the voltage drop between the transmission of the current (I) from an analog switch in the medical device and the return of the current to the medical device through the tissue and lung cavity of the patient. In one or more embodiments, the voltage sensing circuitry is configured to measure the voltage drop between (i) a line adjacent to the line that delivers the current to the distal electrode and (ii) the return current at the surface electrode on the case. In one or more embodiments, in task 420, the voltage sensing circuit utilizes a 4-point measurement (i.e., a Kelvin measurement; a four-electrode measurement; or a tetrapolar measurement) to determine the voltage drop. As described above, measuring the voltage drop in this manner is configured to eliminate (or at least minimize or reduce) voltage drop due to various components of the system (e.g., the resistance of the lead, the nerve cuff, the analog switch, and various metal traces) that are not part of (or attributable to) the transthoracic impedance of the patient, and thereby measure or determine the voltage drop due only to (or substantially only to) the bioimpedance of the patient. That is, the four-point measurement technique is configured to eliminate unwanted interface impedance at the electrodes and the impedance of the connecting lead that are not part of (or attributable to) the transthoracic impedance of the patient.

In the illustrated embodiment, the method 400 also includes a task 430 of determining, by the controller (e.g., MCU) of the medical device, a thoracic impedance of the patient based on the voltage drop determined in task 420. The voltage drop determined in task 420 is a function of the bioimpedance of the patient and the current delivered by the distal electrode, as shown in Equation 1 above. In one or more embodiments, the task 430 of determining the thoracic impedance may be determined utilizing Equation 2 above. As described above, the bioimpedance measured or determined by the controller is a function of (i) the patient's tissue and the volume of air in the patient's thoracic cavity and (ii) the volume of blood in the patient's thoracic cavity. Accordingly, the thoracic impedance measured or determined by the controller in task 430 varies depending on whether the patient is inhaling or exhaling (i.e., the thoracic impedance increases as the airway opens and the thoracic impedance decreases as the airway closes) and whether the patient's heartbeat is in the contraction phase or the expansion phase (i.e., the thoracic impedance increases during the contraction phase of the heartbeat in which the heart pumps blood out and the thoracic impedance decreases during the relaxation phase of the heartbeat in which the heart fills with blood). Accordingly, in one or more embodiments, the thoracic impedance may be utilized to determine the patient's respiratory cycle and/or the extent of opening or closure of the patient's airway.

In the illustrated embodiment, the method 400 also includes a task 440 of delivering electrical stimulation (i.e., neurostimulation) to the patient via the distal electrode at the distal end of the implantable lead based on the thoracic impedance of the patient determined in task 430. The electrical stimulation delivered in 440 may by utilized to treat any medical condition afflicting the patient, such as epilepsy, depression, and/or obstructive sleep apnea (OSA), depending on the location of the nerve cuff in the patient. For instance, in one or more embodiments, the task 440 may include stimulating a nerve of the patient associated with sleep apnea (e.g., the hypoglossal nerve and/or the ansa cervicalis) with the distal electrode based on the thoracic impedance of the patient (determined in task 430) that indicates that the patient is inhaling. That is, the task 440 may include utilizing the thoracic impedance to determine when the patient is inhaling and to stimulate the nerve of the patient when the patient is inhaling. Stimulating the hypoglossal nerve causes the patient's tongue to move, which relieves upper airway obstruction and thereby permits the patient to breath freely. Stimulating the ansa cervicalis nerve pulls the pharyngeal structures of the patient downward, which may relieve the patient of symptoms associated with sleep apnea.

In one or more embodiments, the method 400 may include switching, utilizing time-multiplexing, between the task 440 of delivering the electrical stimulation to the patient with the distal electrode and the task 430 of determining or measuring, in-vivo, the voltage drop of the current that returns to the surface electrode on the can (housing) of the medical device through the patient rather than through the implantable lead.

The system and/or any other relevant devices or components according to embodiments of the present disclosure described herein may be implemented utilizing any suitable hardware, firmware (e.g., an application-specific integrated circuit), software, or a combination of software, firmware, and hardware. For example, the one or more suitable components of the system may be formed on one integrated circuit (IC) chip or on separate IC chips. Further, the one or more suitable components of the system may be implemented on a flexible printed circuit film, a tape carrier package (TCP), a printed circuit board (PCB), or formed on one substrate. Further, the one or more suitable components of the system may be a process or thread, running on one or more processors, in one or more computing devices, executing computer program instructions and interacting with other system components for performing the one or more suitable functionalities described herein. The computer program instructions are stored in a memory which may be implemented in a computing device utilizing a standard memory device, such as, for example, a random access memory (RAM). The computer program instructions may also be stored in other non-transitory computer readable media such as, for example, a flash drive, and/or the like. Also, a person of skill in the art should recognize that the functionality of one or more suitable computing devices may be combined or integrated into a single computing device, or the functionality of a particular computing device may be distributed across one or more other computing devices without departing from the scope of the example embodiments of the present disclosure.

Although some embodiments of the present disclosure have been disclosed herein, the present disclosure is not limited thereto, and the scope of the present disclosure is defined by the appended claims and equivalents thereof.

What is claimed is:

1. An implantable medical system comprising:
a lead comprising a proximal end and a distal end;
a plurality of distal electrodes coupled to the distal end of the lead; and
a medical device coupled to the proximal end of the lead, the medical device comprising:
a housing;
at least one surface electrode on the housing;
an AC current source in the housing;
a multipole analog switch in the housing coupled to the AC current source;
a voltmeter in the housing, the voltmeter comprising a first input coupled to a first distal electrode of the plurality of distal electrodes via the lead, and a second input coupled to the at least one surface electrode on the housing; and
a controller in the housing coupled to the multipole analog switch, the controller being configured to operate the multipole analog switch to selectively change the implantable medical system between a neurostimulation mode and a bioimpedance measurement mode,
wherein in the neurostimulation mode, AC current is delivered from the AC current source and through the lead to a first distal electrode of the plurality of distal electrodes and returned from a second distal electrode of the plurality of distal electrodes to the medical device through the lead, and
wherein in the bioimpedance measurement mode, AC current is delivered from the AC current source through the lead to a third distal electrode of the plurality of distal electrodes and the voltmeter measures a voltage between the third distal electrode and the at least one surface electrode on the housing.

2. The implantable medical system of claim 1, wherein, in the bioimpedance measurement mode, the controller is configured to determine an impedance based on the voltage sensed by the voltmeter.

3. The implantable medical system of claim 2, wherein, in the bioimpedance measurement mode, the third distal electrode of the plurality of distal electrodes is not configured for neurostimulation.

4. The implantable medical system of claim 2, wherein, in the bioimpedance measurement mode, the controller is configured to determine a heart rate based on variations in the impedance.

5. The implantable medical system of claim 4, wherein, in the bioimpedance measurement mode, the controller is configured to determine parameters of a neurostimulation protocol based on the heart rate.

6. The implantable medical system of claim 5, wherein, in the neurostimulation mode, the first distal electrode and the second distal electrode of the plurality of distal electrodes are not configured for sensing of the voltage.

7. The implantable medical system of claim 2, wherein, in the bioimpedance measurement mode, the controller is configured to determine a respiratory cycle based on variations in the impedance.

8. The implantable medical system of claim 7, wherein the respiratory cycle comprises inspiratory and expiratory phases.

9. The implantable medical system of claim 7, wherein, in the bioimpedance measurement mode, the controller is configured to determine parameters of a neurostimulation protocol utilized in the neurostimulation mode based on the respiratory cycle.

10. The implantable medical system of claim 1, wherein the plurality of distal electrodes comprises at least one of a nerve cuff, a helical cuff, paddle electrodes, or an electrode array.

11. The implantable medical system of claim 1, wherein the medical device is configured for implantation in a thoracic region and the plurality of distal electrodes are configured for implantation in a cervical region.

12. The implantable medical system of claim 1, wherein the medical device and the plurality of distal electrodes are configured for implantation in a thoracic region.

13. The implantable medical system of claim 1, wherein the first distal electrode, the second distal electrode, and the third distal electrode are not arranged sequentially.

14. The implantable medical system of claim 1, wherein the AC current has a frequency in a range from approximately 1 kHz to approximately 100 KHz.

15. The implantable medical system of claim 1, wherein the AC current has an amplitude in a range from approximately 0.1 mA to approximately 5 mA.

16. The implantable medical system of claim 1, wherein, in the bioimpedance measurement mode, the voltmeter is configured to sample the voltage at a sampling rate of up to approximately 400 samples per minute.

17. The implantable medical system of claim 2, wherein, in the bioimpedance measurement mode, the controller is configured to de-noise the impedance utilizing an adaptive threshold algorithm.

18. A method of operating an implantable medical system comprising a lead, a plurality of distal electrodes, and a medical device implanted in a patient, the method comprising:
determining a bioimpedance of the patient comprising:
delivering AC current from an AC current source of the medical device through the lead to a third distal electrode of the plurality of distal electrodes;
measuring, utilizing a voltmeter of the medical device, a voltage between the third distal electrode and at least one surface electrode on a housing of the medical device; and
determining, by the implantable medical system, the bioimpedance of the patient based on the voltage; and
neurostimulating the patient by delivering AC current from the AC current source through the lead to a first distal electrode of the plurality of distal electrodes and returning the AC current from a second distal electrode of the plurality of distal electrodes to the medical device through the lead, wherein the neurostimulating is based on the bioimpedance of the patient.

19. The method of claim 18, further comprising switching, utilizing time-multiplexing, between the determining of the bioimpedance of the patient and the neurostimulating of the patient.

20. The method of claim 18, wherein the medical device is implanted in a thoracic region of the patient and the plurality of distal electrodes are implanted in a cervical region of the patient.

21. The method of claim 18, wherein the medical device and the plurality of distal electrodes are implanted in a thoracic region of the patient.
